(19) United States
(12) Patent Application Publication
(10) Pub. No.: US 2024/0407740 A1
(43) Pub. Date: Dec. 12, 2024

(54) INTEGRATED COMPUTED TOMOGRAPHY ROTATING BASE WITH AN INTEGRATED DRIVE SYSTEM

(71) Applicant: GE Precision Healthcare LLC, Wauwatosa, WI (US)

(72) Inventors: Vishwanath Nayak, Bangalore (IN); Saiesh Suryakant Raiker, Margao (IN); Ramachandra Gururaja Rao, Bangalore (IN); Dhaval Pravinbhai Dangashiya, Ahmedabad (IN)

(21) Appl. No.: 18/330,566

(22) Filed: Jun. 7, 2023

(51) Int. Cl. A61B 6/03; A61B 6/00; A61B 6/06

(52) U.S. Cl. CPC A61B 6/035; A61B 6/06; A61B 6/4435

(57) ABSTRACT

A rotating component for a gantry of a computed tomography (CT) imaging system includes a single-piece structure configured to couple to imaging components. The single-piece structure comprises a drive mechanism integrated on the single-piece structure configured to drive rotation of the single-piece structure and the imaging components in response to a driving force.

### BACKGROUND

The subject matter disclosed herein relates to imaging systems and, more particularly, to an integrated computed tomography (CT) rotating base with an integrated drive system.

Non-invasive imaging technologies allow images of the internal structures or features of a patient to be obtained without performing an invasive procedure on the patient. In particular, such non-invasive imaging technologies rely on various physical principles, such as the differential transmission of X-rays through the target volume or the reflection of acoustic waves, to acquire data and to construct images or otherwise represent the observed internal features of the patient.

For example, in computed tomography (CT) and other X-ray based imaging technologies, X-ray radiation spans a subject of interest, such as a human patient, and a portion of the radiation impacts a detector where the image data is collected. In digital X-ray systems a photodetector produces signals representative of the amount or intensity of radiation impacting discrete pixel regions of a detector surface. The signals may then be processed to generate an image that may be displayed for review. In CT imaging systems, a detector array, including a series of detector elements, produces similar signals through various positions as a gantry is displaced around a patient.

A gantry of CT imaging system includes a rotating portion and a stationary portion. In particular, in CT imaging systems the rotating portion of the gantry is conventionally used to spin the X-ray source (e.g., X-ray tube) and detector components around the imaging volume in which the patient is positioned during a scan. The X-ray tube, collimator, and detector form the critical image chain components of the CT imaging system. These subsystems or components mechanically assembled on a CT gantry base (i.e., the rotating portion) are positioned in an X-ray beam path for image formation. The CT gantry base includes a bearing that is separately coupled to a large and heavy platter shaped gear pulley system driven by a motor that causes rotation of the CT gantry base (and the image chain components). All of these components are separately coupled via numerous bolted assemblies and sub-assemblies requiring a great deal of time and effort.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In one embodiment, a rotating component for a gantry of a computed tomography (CT) imaging system is provided. The rotating component includes a single-piece structure configured to couple to imaging components. The single-piece structure includes a drive mechanism integrated on the single-piece structure configured to drive rotation of the single-piece structure and the imaging components in response to a driving force.

In another embodiment, a computed tomography (CT) imaging system is provided. The CT imaging system includes a gantry including a stationary component and a rotating component coupled to the stationary component via a bearing. The rotating component includes a structure configured to couple to imaging components. The structure includes a first annular structure and a second annular structure. The structure also includes a plurality of cross bars disposed between and coupled to both the first annular structure and the second annular structure. The plurality of cross bars are spaced apart relative to each other in a circumferential direction relative to an axis of rotation of the structure. The first annular structure includes a plurality of mounting holes configured to enable the structure to be directly coupled to the bearing without needing to align the structure to the bearing.

In a further embodiment, a rotating component for a gantry of a computed tomography (CT) imaging system is provided. The rotating component includes a single-piece structure configured to couple to imaging components and to rotate about an axis. The single-piece structure also includes a first annular structure and a second annular structure. The single-piece structure also includes a plurality of cross bars disposed between and coupled to both the first annular structure and the second annular structure. The plurality of cross bars are spaced apart relative to each other in a circumferential direction relative to the axis. The single-piece structure further includes an enclosure integrated on the second annular structure and configured for mounting a collimator within the enclosure, mounting an X-ray source on a side of the enclosure opposite from where the collimator is configured to be mounted, and enabling both the X-ray source and the collimator to be coupled to and to be decoupled from the enclosure mutually exclusive of each other.

### BRIEF DESCRIPTION OF THE DRAWINGS

These and other features, aspects, and advantages of the disclosed subject matter will become better understood when the following detailed description is read with reference to the accompanying drawings in which like characters represent like parts throughout the drawings, wherein:

FIG. 1 is a combined pictorial view and block diagram of a computed tomography (CT) imaging system as discussed herein;

FIG. 2 is a perspective view of a CT rotating base, in accordance with aspects of the present disclosure;

FIG. 3 is a front view of the CT rotating base in FIG. 2, in accordance with aspects of present disclosure;

FIG. 4 is back view of the CT rotating base in FIG. 2, in accordance with aspects of the present disclosure;

FIG. 5 is side view of the CT rotating base in FIG. 2, in accordance with aspects of the present disclosure;

FIG. 6 is a side view (e.g., opposite the side in FIG. 5) of the CT rotating base in FIG. 2, in accordance with aspects of the present disclosure;

FIG. 7 is a bottom view of an enclosure of the CT rotating base in FIG. 2, in accordance with aspects of the present disclosure;

FIG. 8 is a perspective view of the enclosure in FIG. 7, in accordance with aspects of the present disclosure;

FIG. 9 is a front view of an extension of the CT rotating base in FIG. 2, in accordance with aspects of the present disclosure;

FIG. 10 is a perspective view of a drive mechanism of the CT rotating base in FIG. 2, in accordance with aspects of the present disclosure;

FIG. 11 is a perspective view of the CT rotating base in FIG. 2 coupled to various components, in accordance with aspects of the present disclosure;

FIG. 12 depicts a stress plot and zoomed in portion of the stress plot performed on the CT rotating base in FIG. 2, in accordance with aspects of the present disclosure;

FIG. 13 depicts a deflection plot for deformation performed on the CT rotating base in FIG. 2, in accordance with aspects of the present disclosure; and

FIG. 14 is a side view of a CT rotating base, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

The present disclosure provides embodiments for an integrated computed tomography) CT rotating base (e.g., drum) with a drive mechanism and mounting interfaces for image chain components (e.g., X-ray tube, collimator, detector) integrated into a single component (e.g., single part, piece, or structure). The CT rotating base is the rotating component of a gantry. The CT rotating base includes a single-piece structure used to house and/or mount multiple components of the imaging chains. The single-piece structure may be manufactured via machining, forging, moulding, casting, weldment, or any three-dimensional (3D) printing process, or any combination of these processes. The CT rotating base also includes a drive mechanism integrated on the single-piece structure configured to drive rotation of the single-piece structure and the imaging components in response to a drive force (e.g., from a pulley or belt coupled to a motor).

The CT rotating base includes a cylindrical shape. In certain embodiments, the rotating base may have a different shape. In particular, the single-piece structure includes a first annular member or portion or structure, a second annular member or portion or structure, and a plurality of cross bars disposed between and coupled to both the first annular structure and the second annular structure. The plurality of cross bars are spaced apart relative to each other in a circumferential direction relative to an axis of rotation of the single-piece structure or CT rotating base. The drive mechanism is located on a surface (e.g., outer surface facing away from the axis of rotation) of an outer perimeter of the first annular structure. In certain embodiments, the drive mechanism may include teeth or grooves. In certain embodiments, the drive mechanism may be flat.

The single-piece structure includes separate mounting structures integrated on the single-piece structure that are configured for mounting the imaging components on the single-piece structure. For example, the single-piece structure includes a plurality of detector mounting structures located on the second annular structure. In particular, a flat extension extends in a radial direction away from the axis of rotation and an outer perimeter of the second annular structure has the plurality of detector mounting structures. Each detector mounting structure is configured for directly mounting (i.e., without an intermediate interface (e.g., detector rail) between the X-ray detector module and the detector mounting structure) of a respective X-ray detector module of an X-ray detector assembly. The single-piece structure also includes another mounting structure (e.g., an enclosure) located on the second annular structure (e.g., in a surface of second annular structure facing away from the first annular structure). The enclosure is configured for mounting the collimator within the enclosure (e.g., on a side of the enclosure facing the detector assembly). The enclosure is also configured for mounting an X-ray source (e.g., X-ray tube) on a side of the enclosure opposite from where the collimator is configured to be mounted (i.e., side facing away from the detector assembly). The enclosure is also configured to enable both the X-ray source and the collimator to be coupled to and to be decoupled from the enclosure mutually exclusive of each other. The modularity of the imaging components (e.g., X-ray tube, collimator, and detector assembly) on the CT rotating base minimizes beam on window (BOW) alignment of the X-ray tube and detector, thus, reducing the overall alignment time at the customer site. In addition, the integration of the mounting structure for the collimator eliminates additional bulky components that are normally coupled to the base to form the collimator housing.

Besides the mounting structures, the CT rotating base has mounting structures for other components. For example, the CT rotating base has a mounting structure (e.g., flat extension portion) integrated on the second annular structure for coupling to a power supply (e.g., electrical housing and power supply) of the CT imaging system. In addition, the CT rotating base includes a plurality of mounting holes located on (and extending through) the first annular structure that enable the single-piece structure to be directly coupled to a bearing of the gantry (which is coupled to a stationary component of the gantry) without needing to align the single-piece structure to the bearing of the gantry (i.e., no need for centering the rotating base with respect to the bearing).

In addition, a profile geometry of the CT rotating base provides for a self-balancing rotor system. In other words, a balance block does not need to be coupled to the rotating component of the gantry for counterbalancing the CT rotating base when all of the imaging components (and other components) are coupled to the CT rotating base. In certain embodiments, one or more balance sheets may be coupled to the single-piece structure to help with the self-balancing to account for part to part weight and center of gravity variation. In certain embodiments, the balance sheets may be separate from the single-piece structure. In certain embodiments, the balance sheets may be integrated with the single-piece structure.

The disclosed CT rotating base significantly reduces the number of bolted joints and assemblies (and other parts) on the rotating base and, thus provides better structural integrity. In addition, due to these reduced number of bolted joints and assemblies, the disclosed rotating base is significantly lighter (e.g., with an approximately 40 percent reduction in weight) than a typical rotating base while still providing a high strength configuration. The configuration of the CT rotating base is configured to uniformly distribute stress with minimal deformation. The disclosed rotating base is scalable in configuration and can be adapted for other CT platforms. For example, a depth of the single-piece structure between the first annular structure and the second annular structure may be adjusted.

The disclosed CT rotating base provides a built-in drive mechanism for many types of drive mechanisms that avoids needing any additional or intermittent separate driven provisions to transmit motion from the drive to the gantry that are typically required with rotating bases. In addition, the unified drive solution for the rotating base provides an entire structure with a plug and play fit that eliminates any misalignments that occur with a drive-driven system while also enhancing motion performance and reliability of the drive system. The disclosed CT rotating base having the integrated drive mechanism eliminates the assembly alignment process of a drive wheel with a gantry rotating base.

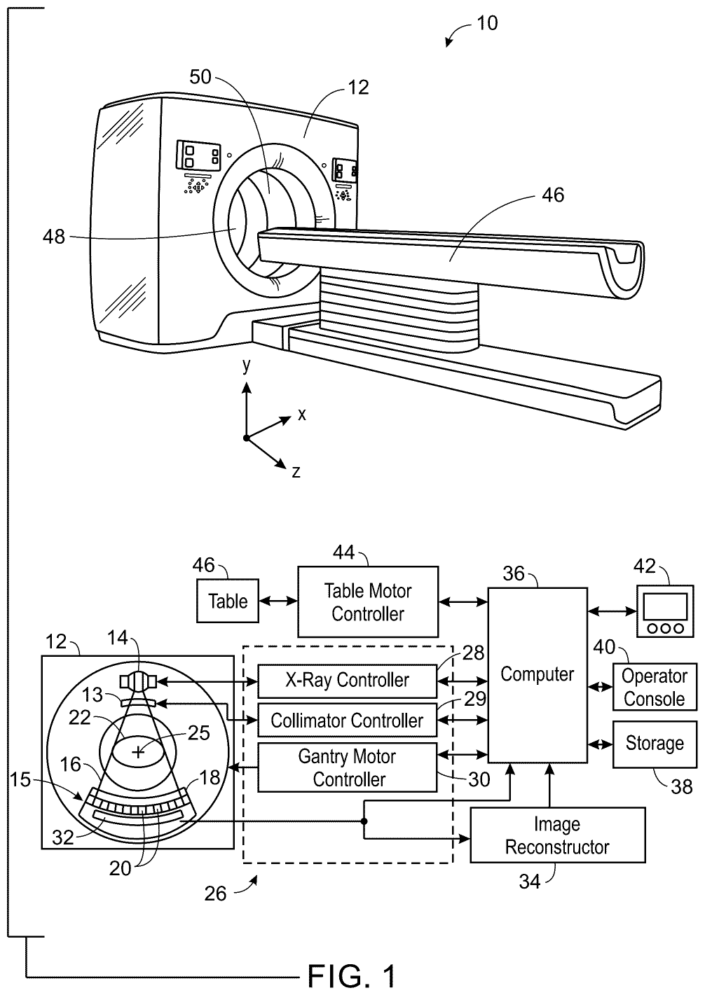

With the preceding in mind and referring to FIG. 1, a computed tomography (CT) imaging system 10 is shown, by way of example. The CT imaging system 10 includes a gantry 12. The gantry 12 has an X-ray source 14 that projects a beam of X-rays 16 toward a detector assembly 15 on the opposite side of the gantry 12. The X-ray source 14 projects the beam of X-rays 16 through a pre-patient collimator assembly 13 that determines the size and shape of the beam of X-rays 16. The detector assembly 15 includes a collimator assembly 18 (a post-patient collimator assembly), a plurality of detector modules 20 (e.g., detector elements or sensors), and data acquisition systems (DAS) 32. The plurality of detector modules 20 detect the projected X-rays that pass through a subject or object 22 being imaged, and DAS 32 converts the data into digital signals for subsequent processing. Each detector module 20 in a conventional system produces an analog electrical signal that represents the intensity of an incident X-ray beam and hence the attenuated beam as it passes through the subject or object 22. During a scan to acquire X-ray projection data, gantry 12 and the components mounted thereon rotate about a center of rotation 25 (e.g., isocenter) so as to collect attenuation data from a plurality of view angles relative to the imaged volume.

Rotation of gantry 12 and the operation of X-ray source 14 are governed by a control system 26 of CT imaging system 10. Control system 26 includes an X-ray controller 28 that provides power and timing signals to an X-ray source 14, a collimator controller 29 that controls a length and a width of an aperture of the pre-patient collimator 13 (and, thus, the size and shape of the beam of X-rays 16), and a gantry motor controller 30 that controls the rotational speed and position of gantry 12. An image reconstructor 34 receives sampled and digitized X-ray data from DAS 32 and performs high-speed image reconstruction. The reconstructed image is applied as an input to a computer 36, which stores the image in a storage device 38. Computer 36 also receives commands and scanning parameters from an operator via console 40. An associated display 42 allows the operator to observe the reconstructed image and other data from computer 36. The operator supplied commands and parameters are used by computer 36 to provide control signals and information to DAS 32, X-ray controller 28, collimator controller 29, and gantry motor controller 30. In addition, computer 36 operates a table motor controller 44, which controls a motorized table 46 to position subject 22 and gantry 12. Particularly, table 46 moves portions of subject 22 through a gantry opening or bore 48.

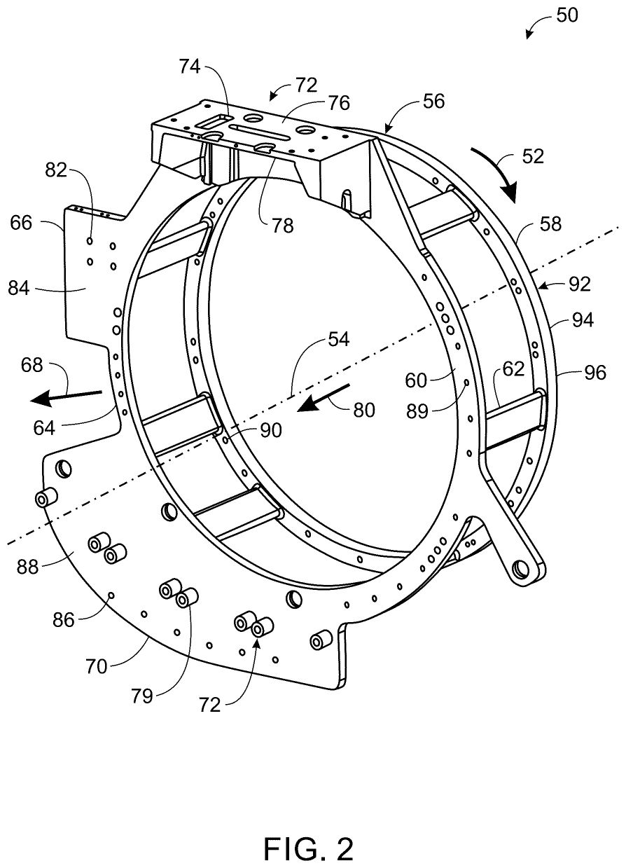

FIGS. 2-6 illustrate different views of a CT rotating base 50 (e.g., drum). The CT rotating base 50 is a rotating component of a gantry (e.g., gantry 12 in FIG. 1) that is configured to be disposed within a housing of the gantry. The CT rotating base 50 is configured to be coupled to a stationary component via a bearing within the housing of the gantry. The CT rotating base 50 is configured to rotate in a circumferential direction 52 about an axis of rotation 54.

The CT rotating base 50 is made of a single-piece structure 56. The single-piece structure 56 may be manufactured via machining, forging, moulding, casting, weldment, or any three-dimensional (3D) printing process, or any combination of these processes. The single-piece structure 56 may made of aluminum, steel, or other metal, or metal alloy. In certain embodiments, the single-piece structure 56 may weigh approximately 56 kilograms (kg). In certain embodiments, the single-piece structure 56 may weigh approximately 40 percent less than a typical CT rotating base.

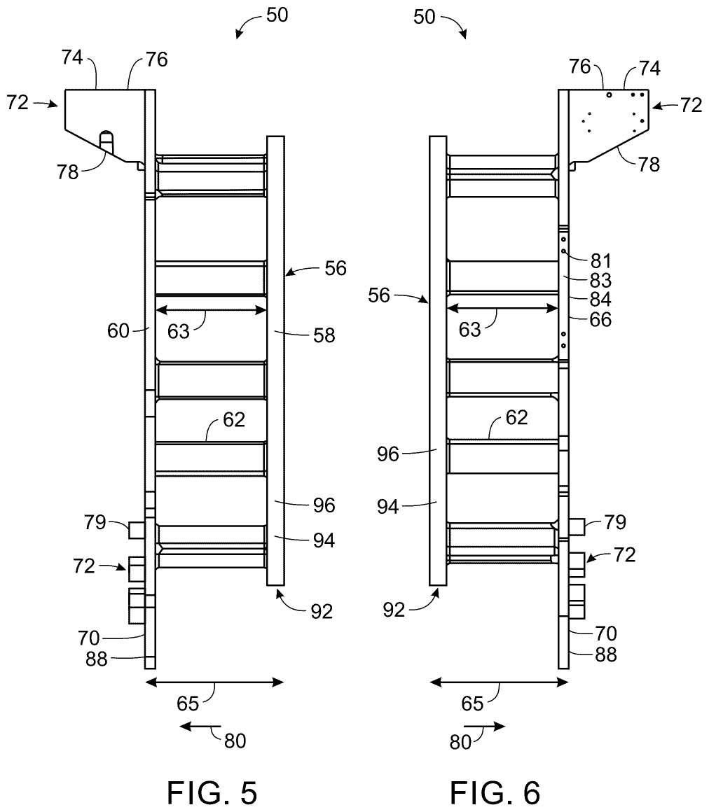

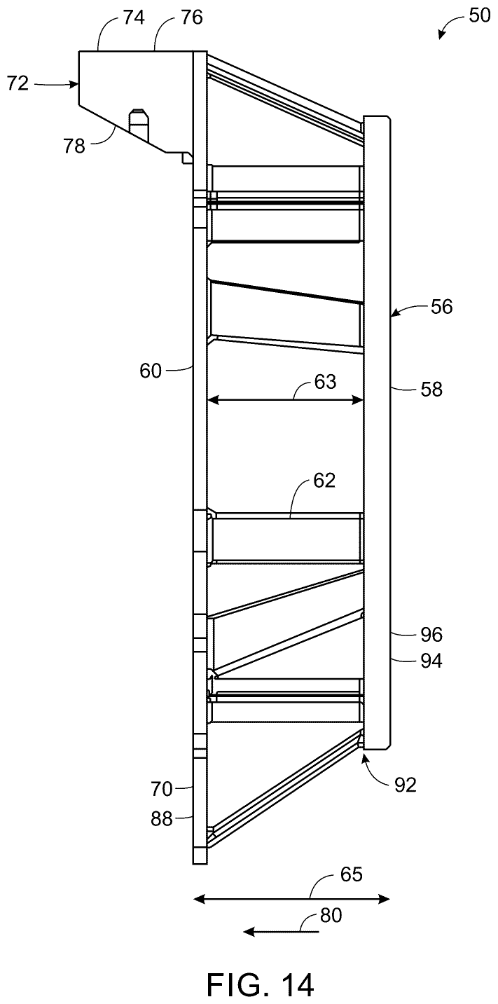

The single-piece structure 56 has a cylindrical shape. The single-piece structure 56 includes a first annular member or portion or structure 58 and a second annular member or portion or structure 60. The single-piece structure 56 also includes a plurality of structural members or cross bars 62 disposed between and coupled to both the first annular structure and the second annular structure. The plurality of cross bars 62 are spaced apart relative to each other in the circumferential direction 52 relative to the axis of rotation 54 of the single-piece structure 56 or the CT rotating base 50. The plurality of cross bars 62 are configured to provide the lowest amount of deflection and the lowest amount of stress. The CT rotating base 50 (i.e., the single-piece structure 56) is scalable in configuration and can be adapted for other CT platforms. A width 63 (e.g., shown in FIGS. 5 and 6) of the plurality of cross bars 62 may vary. In particular, the width 63 of the plurality of cross bars 62 may be adjusted based on the configuration of the CT imaging system. Thus, a depth 65 (e.g., shown in FIGS. 5 and 6) of the single-piece structure 56 in the direction 80 between the first annular structure 58 and the second annular structure 60 may also be adjusted based on the configuration of the CT imaging system. The depth 65 of single-piece structure minimizes the gantry depth. It should be noted that shape and/or arrangement of the CT rotating base 50 and/or the components of the CT rotating base 50 may vary from that depicted in FIGS. 2-6. For example, the location and angle of the cross bars 62 (e.g., relative to an axis of rotation of the CT rotating base 50) may be different as depicted in FIG. 14.

Returning to FIGS. 2-6, the second annular structure 60 includes an outer perimeter 64. The second annular structure 60 includes a first flat extension portion 66 (integral to the single-piece structure 56) that extends in a radial direction 68 (e.g., orthogonal to the axis of rotation 54) away from the axis of rotation 54 and the outer perimeter 64 of the second annular structure 60. The second annular structure 60 also includes a second flat extension portion 70 (integral to the single-piece structure 56) that extends in the radial direction 68 (e.g., orthogonal to the axis of rotation 54) away from the axis of rotation 54 and the outer perimeter 64 of the second annular structure 60.

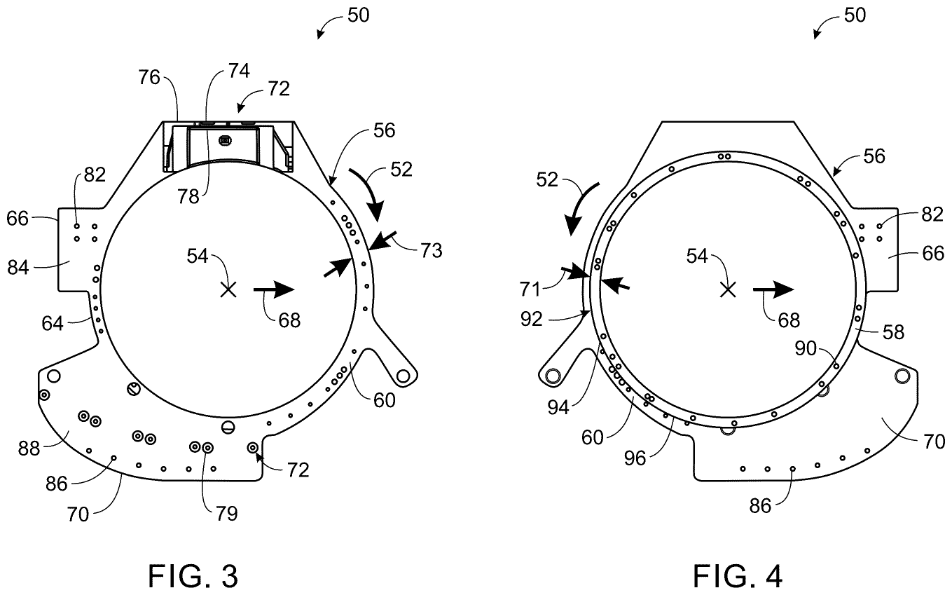

As depicted in FIG. 4, a width 71 in the radial direction 68 of the first annular structure 58 may be constant in the circumferential direction 52. As depicted in FIG. 3, a width 73 in the radial direction 68 of the second annular structure 60 may vary in the circumferential direction 52. As depicted in FIGS. 3 and 4, the width 73 may be same or greater than the width 73 at each corresponding point or location in the circumferential direction 52.

The second annular structure 60 includes separate mounting structures 72 for mounting imaging components to the single-piece structure 56. The separate mounting structures 72 include an enclosure 74 for mounting both an X-ray source (e.g., X-ray tube) and a collimator. The enclosure 74 includes a first side 76 and a second side 78. The enclosure 74 is configured for mounting the collimator within the enclosure 74 on the second side 78 (which faces the detector assembly when coupled to the single-piece structure 56). The enclosure 74 is also configured for mounting the X-ray source on the first side 76 of the enclosure 74 opposite from where the collimator is configured to be mounted (i.e., side facing away from the detector assembly). The enclosure 74 is also configured to enable both the X-ray source and the collimator to be coupled to and to be decoupled from the enclosure 74 mutually exclusive of each other.

The separate mounting structures 72 also include a plurality of detector mounting structures 79 located on the second flat extension portion 70. Each detector mounting structure 79 is configured for direct mounting (i.e., without an intermediate interface (e.g., detector rail) between the X-ray detector module and the detector mounting structure) of a respective X-ray detector module of an X-ray detector assembly. As depicted, the plurality of detector mounting structures 79 extend in a direction 80 (parallel with the axis of rotation 54) away from both the first annular structure 58 and the second flat extension portion 70. The arrangement of the plurality of detector mounting structures 79 may vary from that depicted in FIG. 2. In certain embodiments, the detector mounting structure 79 is configured for indirect mounting of the X-ray detector modules via a detector rail mounted on the detector mounting structure 79.

The modularity of the imaging components (e.g., X-ray tube, collimator, and detector assembly) on the CT rotating base 50 minimizes beam on window (BOW) alignment of the X-ray tube and detector, thus, reducing the overall alignment time at the manufacturing site and the customer site. In addition, the integration of the mounting structure (e.g., enclosure 74) for the collimator eliminates additional bulky components that are normally coupled to the base to form the collimator housing.

The first flat extension portion 66 functions as a mounting structure for a power supply (e.g., electrical housing and power supply) of the CT imaging system to be coupled to the single-piece structure 56. In particular, the first flat extension portion 66 includes a plurality of holes 81 on a surface 83 (as depicted in FIG. 6) of the first flat extension portion 66 facing away from the axis of rotation 54 in the radial direction 68. The plurality of holes enable the power supply to be mounted to the single-piece structure 56. The first flat extension portion 66 also includes a plurality of holes 82 for mounting of a balance sheet on a surface 84 (e.g., facing away from the first annular structure 58). The balance sheet may assist the CT rotating base 50 in self balancing when rotating with all of components coupled to it.

The second flat extension portion 70 also serves as a mounting structure for additional components. As depicted in FIG. 2, the second flat extension portion 70 includes a plurality of holes 86 for coupling a plate on a surface 88 (e.g., facing away from the first annular structure 58) of the second flat extension portion 70. The plate may be coupled to additional components (e.g., DAS).

The second annular structure 60 includes a plurality of holes 89 to enable mounting of a generator (e.g., high voltage generator) to the single-piece structure 56. In particular, the plurality of holes 89 enable a bracket to be coupled to the second annular structure 60 and the generator is coupled to the bracket. The generator provides power to the X-ray source.

The first annular structure 58 includes a plurality of holes 90 (e.g., mounting holes) spaced apart along the first annular structure 58 in the circumferential direction 52. The plurality of holes 90 enable (via the first annular structure 58) the single-piece structure 56 to be directly coupled to a bearing of the gantry (which is coupled to a stationary component of the gantry) without needing to align the single-piece structure 56 to the bearing of the gantry (i.e., no need for centering the rotating base 50 with respect to the bearing).

The single-piece structure 56 also includes a drive mechanism 92 integrated on the single-piece structure 56 that is configured to drive rotation of the single-piece structure 56 and the imaging components in response to a drive force (e.g., from a pulley or belt coupled to a motor). The drive mechanism 92 is located on a surface 94 (e.g., outer surface facing away from the axis of rotation 54) of an outer perimeter 96 of the first annular structure 58. As depicted, the surface 94 of the drive mechanism 92 is flat or smooth. In certain embodiments, the drive mechanism 92 may include grooves. In certain embodiments, the drive mechanism 92 may include teeth (e.g., gear teeth). The built-in drive mechanism avoids needing any additional or intermittent separate driven provisions to transmit motion from the drive to the gantry that are typically required with rotating bases. In addition, the unified drive solution for the rotating base 50 provides an entire structure with a plug and play fit that eliminates any misalignments that occur with a drive-driven system while also enhancing motion performance and reliability of the drive system. Also, the integrated drive mechanism 92 eliminates the assembly alignment process of a drive wheel with a gantry rotating base.

A profile geometry of the single-piece structure 56 provides for a self-balancing rotor system. In other words, a balance block does not need to be coupled to the rotating component of the gantry for counterbalancing the CT rotating base 50 when all of the imaging components (and other components) are coupled to the CT rotating base 50. In certain embodiments, one or more balance sheets may be coupled to the single-piece structure 56 to help with the self-balancing. In certain embodiments, the balance sheets may be separate from the single-piece structure 56. In certain embodiments, the balance sheets may be integrated with the single-piece structure 56.

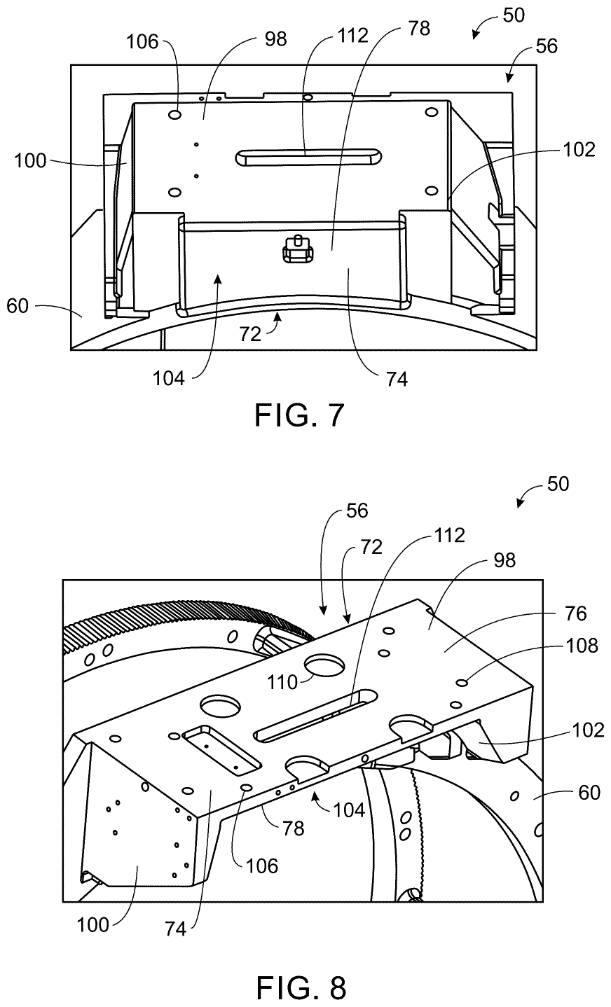

FIGS. 7 and 8 are different views of the enclosure 74 of the CT rotating base 50 in FIG. 2. As noted, the enclosure 74 is configured for mounting both an X-ray source (e.g., X-ray tube) and a collimator. The enclosure 74 includes a top wall 98 having the first side 76 and the second side 78. The enclosure 74 also includes a pair of walls 100, 102 flanking the top wall 98. The pair of walls 100, 102 extend in the radial direction 68 toward the axis of rotation 54 (see FIG. 2). The walls 98, 100, 102 define a space 104. The enclosure 74 is configured for mounting the collimator within space 104 of the enclosure 74 on the second side 78 (which faces the detector assembly when coupled to the single-piece structure 56). The enclosure 74 includes a plurality of holes 106 for coupling the collimator.

The enclosure 74 is also configured for mounting the X-ray source on the second side 78 of the enclosure 74 opposite from where the collimator is configured to be mounted (i.e., side facing away from the detector assembly). The wall 98 includes a plurality of holes 108 for coupling the X-ray source to the first side 76. The wall 98 also includes a plurality of recesses 110 on the second side 78 for receiving the X-ray source. The wall 98 further includes an aperture 112 for the X-ray beams emitted by the X-ray source. The enclosure 74 is also configured to enable both the X-ray source and the collimator to be coupled to and to be decoupled from the enclosure 74 mutually exclusive of each other.

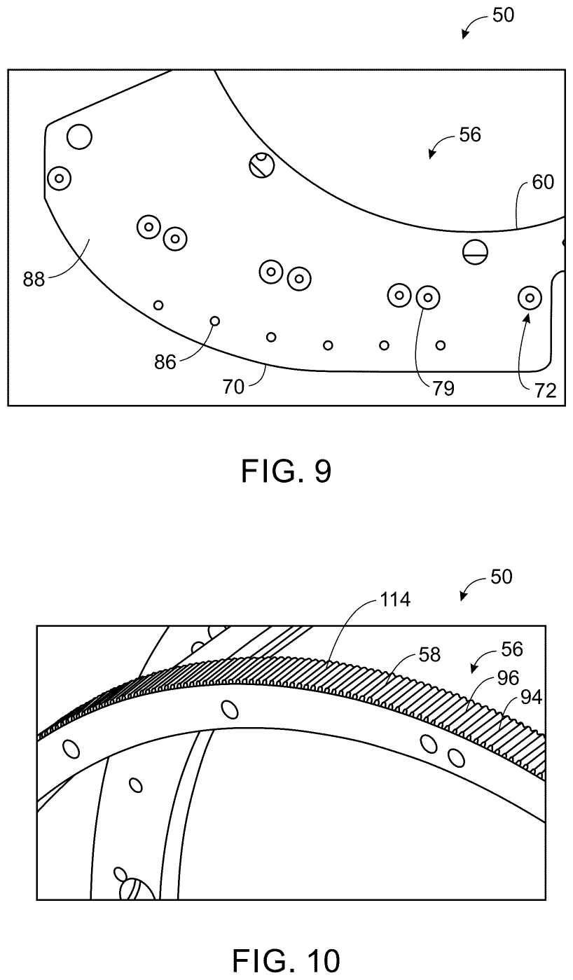

FIG. 9 is a front view of an extension (second flat extension portion 70) of the CT rotating base 50 in FIG. 2. As noted above, the second flat extension portion 70 (integral to the single-piece structure 56) extends in the radial direction 68 (e.g., orthogonal to the axis of rotation 54) away from the axis of rotation 54 and the outer perimeter 64 of the second annular structure 60. The second flat extension portion 70 includes the plurality of detector mounting structures 79 located on the second flat extension portion 70. Each detector mounting structure 79 is configured for directly mounting (i.e., without an intermediate interface (e.g., detector rail) between the X-ray detector module and the detector mounting structure) of a respective X-ray detector module of an X-ray detector assembly. As depicted in FIG. 2, the plurality of detector mounting structures 79 extend in the direction 80 (parallel with the axis of rotation 54) away from both the first annular structure 58 and the second flat extension portion 70. As noted above, the second flat extension portion 70 also serves as a mounting structure for additional components. The second flat extension portion 70 includes the plurality of holes 86 for coupling a plate on a surface 88 (e.g., facing away from the first annular structure 58) of the second flat extension portion 70. The plate may be coupled to additional components (e.g., DAS).

FIG. 10 is a perspective view of the drive mechanism 92 of the CT rotating base 50 in FIG. 2. As noted above, the single-piece structure 56 also includes the drive mechanism 92 integrated on the single-piece structure 56. The drive mechanism is configured to drive rotation of the single-piece structure 56 and the imaging components in response to a drive force (e.g., from a pulley or belt coupled to a motor). The drive mechanism 92 is located on the surface 94 (e.g., outer surface facing away from the axis of rotation 54) of the outer perimeter 96 of the first annular structure 58. As depicted, the drive mechanism 92 includes teeth 114 (e.g., gear teeth). In certain embodiments, the drive mechanism 92 may include grooves. In certain embodiments, the drive mechanism 92 may be flat (e.g., as depicted in FIGS. 2-6).

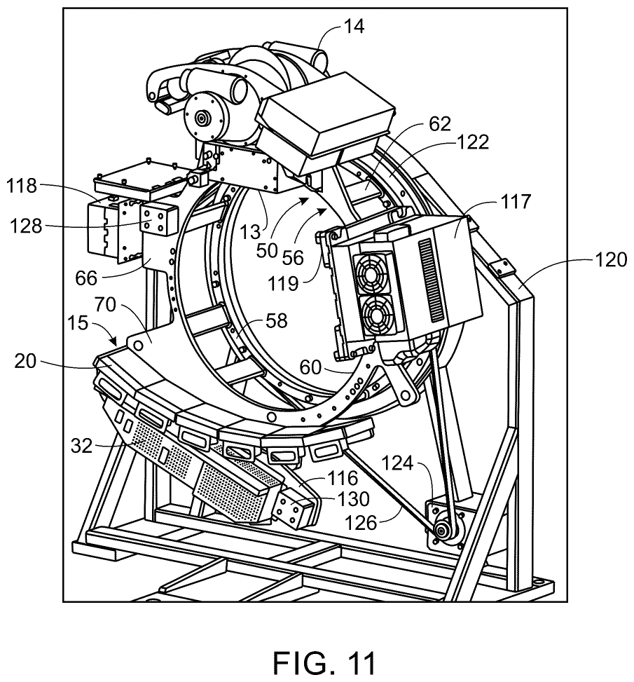

FIG. 11 is a perspective view of the CT rotating base 50 in FIG. 2 coupled to various components. As depicted in FIG. 11, the collimator 13 (e.g., pre-patient collimator assembly) is disposed within the enclosure 74. The enclosure 74 also includes the X-ray source 14 (e.g., X-ray tube) mounted to the enclosure 74. As depicted, the collimator 13 is mounted within the enclosure 74 on one side (e.g., second side 78 in FIGS. 7 and 8). The X-ray source 14 is mounted on top of the enclosure 74 on an opposite side (e.g., first side 76 in FIGS. 7 and 8). As noted above, the enclosure 74 is also configured to enable both the X-ray source 14 and the collimator 13 to be coupled to and to be decoupled from the enclosure 74 mutually exclusive of each other.

Also, as depicted in FIG. 11, the detector assembly 15 is directly coupled to the surface 88 on the second flat extension portion 70. In particular, a plurality of detector modules 20 of the detector assembly 15 is directly coupled to the surface 88 via detector mounting structures (e.g., detector mounting structures 79 in FIG. 2) on the second flat extension portion 70. The detector modules 20 are directly coupled to the second extension portion 70 without an intermediate interface (e.g., detector rail) between the detector modules 20 and second extension portion 70.

Further, as depicted in FIG. 11, a plate 116 is coupled to the second flat extension portion 70 (e.g., via plurality of holes 86 in FIG. 9). The DAS 32 is mounted on the plate 116. Even further, a power supply 118 (e.g., electrical housing and power supply) of the CT imaging system is mounted on the first flat extension portion 66 (e.g., the plurality of holes 81 on the surface 83 in FIG. 6).

Still further, as depicted in FIG. 11, a generator 117 (e.g., high voltage generator) is mounted on the second annular structure 60. In particular, the generator 117 is coupled to the second annular structure 60 via a bracket 119. The bracket 119 is directly coupled to the second annular structure 60.

As depicted, the single-piece structure 56 of the CT rotating base 50 is coupled to a stationary component 120 via a bearing 122. In particular, the first annular structure 58 is coupled to the bearing 122 (e.g., via the holes 90 in FIG. 2). The plurality of holes 90 enable (via the first annular structure 58) the single-piece structure 56 to be directly coupled to the bearing 122 without needing to align the single-piece structure 56 to the bearing 122 (i.e., no need for centering the CT rotating base 50 with respect to the bearing 122).

As depicted, a motor 124 is coupled to the stationary component 120. A belt or pulley 126 is coupled to the motor 124 and disposed about the drive mechanism 92. The drive mechanism 92 drives rotation of the single-piece structure 56 and the imaging components in response to a drive force (e.g., from the pulley or belt 126 coupled to the motor 124).

A profile geometry of the single-piece structure 56 provides for a self-balancing rotor system. In other words, a balance block does not need to be coupled to the rotating component 120 of the gantry for counterbalancing the CT rotating base 50 when all of the imaging components (and other components) are coupled to the CT rotating base 50. In certain embodiments, one or more balance sheets may be coupled to the single-piece structure 56 (e.g., directly or indirectly) to help with the self-balancing. As depicted in FIG. 11, a first balance sheet 128 is coupled to the first flat extension portion 66 (e.g., via holes 86 in FIG. 2). Also, a second balance sheet 130 is coupled to the plate 116 (which is coupled to the second flat extension portion 70). In certain embodiments, the balance sheets may be separate from the single-piece structure 56. In certain embodiments, the balance sheets (e.g., balance sheet 128) may be integrated with or on the single-piece structure 56.

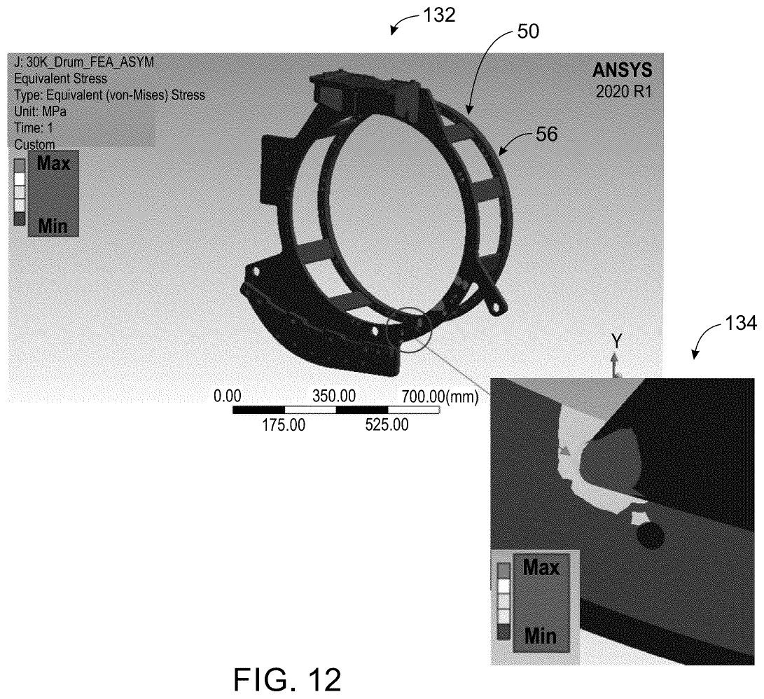

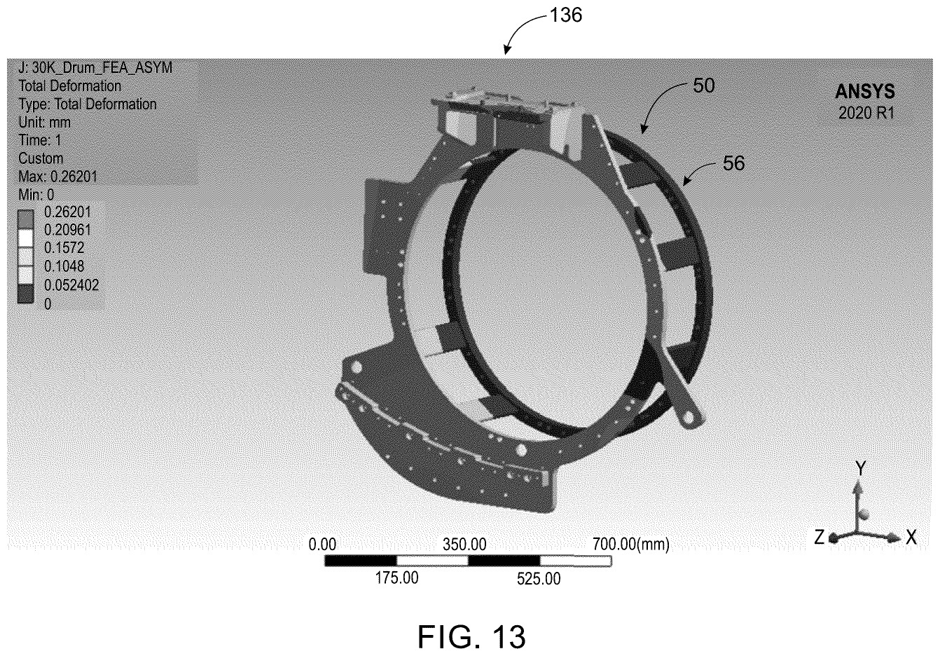

The configuration of the CT rotating base 50 is configured to uniformly distribute stress with minimal deformation. In particular, the plurality of cross bars 62 are configured to provide the lowest amount of deflection and the lowest amount of stress. FIG. 12 depicts a stress plot 132 and zoomed in portion 134 of the stress plot 132 performed on the CT rotating base 50 in FIG. 2. As depicted, the stress is uniformly distributed across the single-piece structure 56 of the CT rotating base 50. FIG. 13 depicts a deflection plot 136 for deformation performed on the CT rotating base 50 in FIG. 2. As depicted, minimal deformation occurs in the single-piece structure 56.

Technical effects of the disclosed embodiments include providing an integrated CT rotating base with a drive mechanism and mounting interfaces for image chain components (e.g., X-ray tube, collimator, detector) integrated into a single component (e.g., single part, piece, or structure). Technical effects of the disclose embodiments also include a significant reduction (e.g., approximately 40 percent reduction) in the rotating portion of the CT gantry. Technical effects of the disclosed embodiments further include a reduction in time and effort during assembly (e.g., by eliminating run out alignment of a driven pulley with respect to a center of rotation). Technical effects of the disclosed embodiments still further include enabling the X-ray tube and the collimator assembly to be assembled and disassembled to the CT rotating base mutually exclusive of each other. Technical effects of the disclosed embodiments yet further include improving structural integrity of the CT rotating base (e.g., via reduction in the number of bolted joints and assemblies). Technical effects of the disclosed embodiments further include providing for direct detector module mounting on the CT rotating base (i.e., without an interfacing component).

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function]. . . ” or “step for [perform]ing [a function]. . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A rotating component for a gantry of a computed tomography (CT) imaging system, comprising:
a single-piece structure configured to couple to imaging components, wherein the single-piece structure comprises a drive mechanism integrated on the single-piece structure configured to drive rotation of the single-piece structure and the imaging components in response to a driving force.

2. The rotating component of claim 1, wherein the single-piece structure comprises separate mounting structures integrated on the single-piece structure and configured for mounting the imaging components on the single-piece structure.

3. The rotating component of claim 2, wherein the imaging components comprise an X-ray source, a collimator, and an X-ray detector assembly.

4. The rotating component of claim 3, wherein the separate mounting structures comprise a plurality of detector mounting structures, wherein each detector mounting structure of the plurality of detector mounting structures is configured for mounting of an X-ray detector module of the X-ray detector assembly.

5. The rotating component of claim 3, wherein the separate mounting structures comprise an enclosure configured for mounting the collimator within the enclosure.

6. The rotating component of claim 5, wherein the enclosure is configured for mounting the X-ray source on a side of the enclosure opposite from where the collimator is configured to be mounted.

7. The rotating component of claim 6, wherein the enclosure is configured to enable both the X-ray source and the collimator to be coupled to and to be decoupled from the enclosure mutually exclusive of each other.

8. The rotating component of claim 1, wherein the single-piece structure is configured for mounting a power supply of the CT imaging system.

9. The rotating component of claim 1, wherein the single-piece structure is configured for self-balancing when rotated.

10. The rotating component of claim 1, wherein the single-piece structure comprises a first annular structure, a second annular structure, and a plurality of cross bars disposed between and coupled to both the first annular structure and the second annular structure, wherein the plurality of cross bars are spaced apart relative to each other in a circumferential direction relative to an axis of rotation of the single-piece structure.

11. The rotating component of claim 10, wherein the drive mechanism is located on a surface of an outer perimeter of the first annular structure.

12. The rotating component of claim 10, wherein the first annular structure comprises a plurality of mounting holes configured to enable the single-piece structure to be directly coupled to a bearing of the gantry without needing to align the single-piece structure to the bearing of the gantry.

13. A computed tomography (CT) imaging system, comprising:
a gantry comprising a stationary component and a rotating component coupled to the stationary component via a bearing, wherein the rotating component comprises a structure configured to couple to imaging components, and wherein the structure comprises:
a first annular structure;
a second annular structure; and
a plurality of cross bars disposed between and coupled to both the first annular structure and the second annular structure, wherein the plurality of cross bars are spaced apart relative to each other in a circumferential direction relative to an axis of rotation of the structure; and
wherein the first annular structure comprises a plurality of mounting holes configured to enable the structure to be directly coupled to the bearing without needing to align the structure to the bearing.

14. The CT imaging system of claim 13, wherein the structure comprises a single-piece structure.

15. The CT imaging system of claim 14, wherein the single-piece structure comprises a drive mechanism integrated on the single-piece structure configured to drive rotation of the single-piece structure and the imaging components in response to a driving force.

16. The CT imaging system of claim 15, wherein the drive mechanism is located on a surface of an outer perimeter of the first annular structure.

17. The CT imaging system of claim 14, wherein the single-piece structure comprises separate mounting structures integrated on the single-piece structure and configured for mounting the imaging components on the single-piece structure, wherein the imaging components comprise an X-ray source, a collimator, and an X-ray detector assembly.

18. The CT imaging system of claim 17, wherein the separate mounting structures are located on the second annular structure.

19. The CT imaging system of claim 18, wherein the separate mounting structures comprise a plurality of detector mounting structures, wherein each detector mounting structure of the plurality of detector mounting structures is configured for mounting of an X-ray detector module of the X-ray detector assembly.

20. A rotating component for a gantry of a computed tomography (CT) imaging system, comprising:
a single-piece structure configured to couple to imaging components and to rotate about an axis, wherein the single-piece structure comprises:
a first annular structure;
a second annular structure;
a plurality of cross bars disposed between and coupled to both the first annular structure and the second annular structure, wherein the plurality of cross bars are spaced apart relative to each other in a circumferential direction relative to the axis; and
an enclosure integrated on the second annular structure and configured for mounting a collimator within the enclosure, mounting an X-ray source on a side of the enclosure opposite from where the collimator is configured to be mounted, and enabling both the X-ray source and the collimator to be coupled to and to be decoupled from the enclosure mutually exclusive of each other.
